(19) United States
(12) Patent Application Publication
(10) Pub. No.: US 2026/0232322 A1
(43) Pub. Date: Aug. 13, 2026

(54) DEVICES FOR TREATMENT OF VASCULAR DEFECTS

(71) Applicant: MICROVENTION, INC., Aliso Viejo, CA (US)

(72) Inventor: Todd Hewitt, Laguna Niguel, CA (US)

(21) Appl. No.: 18/860,828

(22) PCT Filed: May 1, 2023
(86) PCT No.: PCT/US23/20560

§ 371 Date: Oct. 28, 2024

Related U.S. Application Data
(60) Provisional application No. 63/337,558, filed on May 2, 2022.

(51) Int. Cl. A61B 17/12

(52) U.S. Cl. CPC A61B 17/12113; A61B 17/12172; A61B 2017/1205

(57) ABSTRACT

Devices and methods for treatment of a patient's vasculature are described. Embodiments may include a permeable shell having a first layer, a second layer, a proximal end, a distal end, and a transitional section connecting the first and second layer. The first and second layers are formed from a plurality of elongate filaments that are woven together to form a mesh. Each of the plurality of elongate filaments have a first end and a second end, and the first and second ends of each of the plurality of elongate filaments may be gathered at the proximal end of the permeable shell in a proximal band. The transitional section of the permeable shell includes a first end portion connected to a distal end of the first layer and a second end portion connected to a distal end of the second layer. In some embodiments, the second end portion may be inverted.

### CROSS-REFERENCE TO RELATED APPLICATIONS

This application is a national stage patent application under 35 U.S.C. § 371 of International Application No. PCT/US 23/20560, filed May 1, 2023, which claims priority to U.S. Provisional Application Ser. No. 63/337,558, filed May 2, 2022, all of which are hereby expressly incorporated by reference in their entireties for all purposes.

### STATEMENT REGARDING FEDERALLY SPONSORED RESEARCH OR DEVELOPMENT

Not applicable.

### BACKGROUND OF THE INVENTION

The mammalian circulatory system is comprised of a heart, which acts as a pump, and a system of blood vessels that transport the blood to various points in the body. Due to the force exerted by the flowing blood on the blood vessel the blood vessels may develop a variety of vascular defects. One common vascular defect known as an aneurysm is a result of an abnormal widening of the blood vessel. Typically, vascular aneurysms are formed as a result of the weakening of the wall of a blood vessel and subsequent ballooning and expansion of the vessel wall. If, for example, an aneurysm is present within an artery of the brain, and the aneurysm should burst with resulting cranial hemorrhaging, death could occur.

Surgical techniques for the treatment of cerebral aneurysms typically involve a craniotomy requiring creation of an opening in the skull of the patient through which the surgeon can insert instruments to operate directly on the patient's brain. For some surgical approaches, the brain must be retracted to expose the parent blood vessel from which the aneurysm arises.

Once access to the aneurysm is gained, the surgeon places a clip across the neck of the aneurysm thereby preventing arterial blood from entering the aneurysm. Upon correct placement of the clip, the aneurysm should be obliterated in a matter of minutes. Surgical techniques may be effective treatment for many aneurysms. Unfortunately, surgical techniques for treating these types of conditions include major invasive surgical procedures which often require extended periods of time under anesthesia involving high risk to the patient. Such procedures thus require that the patient be in generally good physical condition in order to be a candidate for such procedures.

Various alternative and less invasive procedures have been used to treat cerebral aneurysms without resorting to major surgery. One approach to treating aneurysms without the need for invasive surgery involves the placement of sleeves or stents into the vessel and across the region where the aneurysm occurs. Such flow diverter devices maintain blood flow through the vessel while reducing blood pressure applied to the interior of the aneurysm. Certain types of stents are expanded to the proper size by inflating a balloon catheter, referred to as balloon expandable stents, while other stents are designed to elastically expand in a self-expanding manner. Some stents are covered typically with a sleeve of polymeric material called a graft to form a stent-graft. Stents and stent-grafts are generally delivered to a preselected position adjacent a vascular defect through a delivery catheter. In the treatment of cerebral aneurysms, covered stents or stent-grafts have seen very limited use due to the likelihood of inadvertent occlusion of small perforator vessels that may be near the vascular defect being treated.

In addition, current uncovered stents are generally not sufficient as a stand-alone treatment. In order for stents to fit through the microcatheters used in small cerebral blood vessels, their density is usually reduced such that when expanded, there is only a small amount of stent structure bridging the aneurysm neck. Thus, they do not block enough flow to cause clotting of the blood in the aneurysm and are thus generally used in combination with vaso-occlusive devices, such as the coils discussed above, to achieve aneurysm occlusion.

Some procedures involve the delivery of embolic or filling materials into an aneurysm. The delivery of such vaso-occlusion devices or materials may be used to promote hemostasis or fill an aneurysm cavity entirely. Vaso-occlusion devices may be placed within the vasculature of the human body, typically via a catheter, either to block the flow of blood through a vessel with an aneurysm through the formation of an embolus or to form such an embolus within an aneurysm stemming from the vessel. A variety of implantable, coil-type vaso-occlusion devices are known. The coils of such devices may themselves be formed into a secondary coil shape, or any of a variety of more complex secondary shapes. Vaso-occlusive coils are commonly used to treat cerebral aneurysms but suffer from several limitations including poor packing density, compaction due to hydrodynamic pressure from blood flow, poor stability in wide-necked aneurysms, and complexity and difficulty in the deployment thereof as most aneurysm treatments with this approach require the deployment of multiple coils. Coiling is less effective at treating certain physiological conditions, such as wide neck cavities (e.g., wide neck aneurysms) because there is a greater risk of the coils migrating out of the treatment site. The procedure typically also includes a balloon or stent placed adjacent the aneurysm location to reduce the risk of coils falling out of the aneurysm and migrating elsewhere. Even with these additional devices in use, there is still a risk of the coils sticking out of the aneurysm.

A number of aneurysm neck bridging devices with defect spanning portions or regions have been attempted, however, none of these devices have had a significant measure of clinical success or usage. A major limitation in their adoption and clinical usefulness is the inability to position the defect spanning portion to assure coverage of the neck. Existing stent delivery systems that are neurovascular compatible (i.e., deliverable through a microcatheter and highly flexible) do not have the necessary rotational positioning capability. Another limitation of many aneurysm bridging devices described in the prior art is the poor flexibility. Cerebral blood vessels are tortuous, and a high degree of flexibility is required for effective delivery to most aneurysm locations in the brain.

What has been needed are devices and methods for delivery and use in small and tortuous blood vessels that can substantially block the flow of blood into an aneurysm, such as a cerebral aneurysm, with a decreased risk of inadvertent aneurysm rupture or blood vessel wall damage. In addition, what has been needed are methods and devices suitable for blocking blood flow in cerebral aneurysms over an extended period of time without a significant risk of deformation, compaction, or dislocation.

Intrasaccular occlusive devices are part of a newer type of occlusion device used to treat various intravascular conditions including aneurysms. They are often more effective at treating these wide neck conditions, or larger treatment areas. The intrasaccular devices comprise a structure that sits within the aneurysm and provides an occlusive effect at the neck of the aneurysm to help limit blood flow into the aneurysm. The device includes a relatively conformable structure that sits within the aneurysm helping to occlude all or a portion of the aneurysm. Intrasaccular devices typically conform to the shape of the treatment site. These devices also occlude the cross section of the neck of the treatment site/aneurysm, thereby promoting clotting and causing thrombosis and closing of the aneurysm over time. In larger aneurysms, however, there is a risk of compaction where the intrasaccular device can migrate into the aneurysm and leave the neck region.

Occlusive devices should have certain characteristics. First, the implants should be deliverable with low force typically used). Second, the implants should have a high enough radial forces to fully deploy and remain positioned within the aneurysm or parent artery after detachment (not migrate). Third, the implants need to have high metal surface area to promote flow stasis, which is a reduction of blood flow, so as to promote stagnation and clotting.

The following embodiments describe multiple layer implants that achieve the desired characteristics.

### SUMMARY OF THE INVENTION

An occlusion device is described that is used to treat a variety of conditions, including aneurysms and neurovascular aneurysms, in particular, bifurcation aneurysms. In some embodiments, the occlusion device is configured as an intrasaccular device.

In many embodiments, a device for treatment of a patient's cerebral aneurysm includes a permeable shell comprising first layer, a second layer, a proximal end, a distal end, and an elongate section connecting the first and second layer, wherein the permeable shell has a radially constrained elongated state configured for delivery within a catheter lumen and an expanded state with a longitudinally shortened configuration relative to the radially constrained state, wherein the first and second layers are formed from a plurality of elongate filaments that are woven together to form a mesh, wherein each of the plurality of elongate filaments have a first end and a second end, wherein the first and second ends of each of the plurality of elongate filaments are gathered at the proximal end of the permeable shell in a proximal band, and wherein the elongate section of the permeable shell comprises an intermediate portion of the plurality of filaments, and wherein the intermediate portion of the plurality of filaments is not surrounded by a band.

In some embodiments, the proximal band is the only band in the device.

In some embodiments, the second layer is an inner layer of the device and the first layer is an outer layer of the device.

In some embodiments, the elongate section comprises a height that is less that about ¼ of a total height of the permeable shell.

In some embodiments, in an unconstrained configuration, an expanded shape of the first layer is different than an expanded shape of the second layer.

In some embodiments, in an unconstrained configuration, an outer surface of a distal region of an expanded shape of the first layer has a substantially frustoconical shape.

In some embodiments, in an unconstrained configuration, the second layer comprises an expanded shape comprising a top surface that defines a plane that is substantially perpendicular to a vertical axis of the permeable shell. In some embodiments, in an unconstrained configuration, an inner surface of a distal portion of the expanded shape of the first layer does not contact at least about 50% of a first area defined by the plane of the top surface. In some embodiments, in an unconstrained configuration, the expanded shape of the second layer is substantially barrel shape.

In some embodiments, in an unconstrained configuration, the second layer comprises an expanded shape comprising a proximal portion having a substantially frustoconical shape, wherein the proximal portion of having the substantially frustoconical shape defines a second area. In some embodiments, in an unconstrained configuration, an inner surface of a proximal portion of the expanded shape of the first layer does not contact at least about 50% of the second area.

In some embodiments, the mesh is inverted at the distal end of the permeable shell to form the first and second layers of the permeable shell.

In some embodiments, the mesh is a tubular mesh having an inner surface and an outer surface, wherein an outer surface of the first layer is the inner surface of the tubular mesh.

In some embodiments, in an unconstrained configuration, in an expanded, heat-set state, the permeable mesh further comprises a distal void space in a distal region of the permeable shell and a proximal void space in the proximal region of the permeable shell. In some embodiments, in an unconstrained configuration, the distal void space is larger than the proximal void space. In some embodiments, in an unconstrained configuration, the distal void space is defined by a top surface of the expanded state of the second layer, an outer surface of the elongate section, and an inner surface of a distal region of the first layer. In some embodiments, in an unconstrained configuration, the proximal void space is defined by a bottom surface of an expanded state of the second layer and an inner surface of a proximal region of the first layer.

In many embodiments, a method for treating a cerebral aneurysm having an interior cavity and a neck includes the steps of: advancing an implant in a microcatheter to a region of interest in a cerebral artery, wherein the implant comprises: a permeable shell comprising first layer, a second layer, a proximal end, a distal end, and an elongate section connecting the first and second layer, wherein the permeable shell has a radially constrained elongated state configured for delivery within a lumen of the microcatheter and an expanded state with a longitudinally shortened configuration relative to the radially constrained state, wherein the first and second layers are formed from a plurality of elongate filaments that are woven together to form a mesh, wherein each of the plurality of elongate filaments have a first end and a second end, wherein the first and second ends of each of the plurality of elongate filaments are gathered at the proximal end of the permeable shell in a proximal band, and wherein the elongate section of the permeable shell comprises an intermediate portion of the plurality of filaments, and wherein the intermediate portion of the plurality of filaments is not surrounded by a band; deploying the implant within the cerebral aneurysm, wherein the permeable shell expands to the expanded state in the interior cavity of the aneurysm; and withdrawing the microcatheter from the region of interest after deploying the implant.

In some embodiments, the proximal band is the only band in the device.

In some embodiments, the second layer is an inner layer of the device and the first layer is an outer layer of the device.

In some embodiments, the mesh is inverted at the distal end of the permeable shell to form the first and second layers of the permeable shell.

In some embodiments, the mesh is a tubular mesh having an inner surface and an outer surface, wherein an outer surface of the first layer is the inner surface of the tubular mesh.

In some embodiments, in an unconstrained configuration, in an expanded, heat-set state, the permeable mesh further comprises a distal void space in a distal region of the permeable shell and a proximal void space in the proximal region of the permeable shell. In some embodiments, in an unconstrained configuration, the distal void space is larger than the proximal void space.

In some embodiments, in an unconstrained configuration, the distal void space is defined by a top surface of the expanded state of the second layer, an outer surface of the elongate section, and an inner surface of a distal region of the first layer.

In some embodiments, in an unconstrained configuration, the proximal void space is defined by a bottom surface of an expanded state of the second layer and an inner surface of a proximal region of the first layer.

In many embodiments, a device for treatment of a patient's cerebral aneurysm, includes: a permeable shell comprising a first layer, a second layer, a proximal end, a distal end, and a transitional section connecting the first and second layer, wherein the permeable shell has a radially constrained elongated state configured for delivery within a catheter lumen and an expanded state with a longitudinally shortened configuration relative to the radially constrained state, wherein the first and second layers are formed from a plurality of elongate filaments that are woven together to form a mesh, wherein each of the plurality of elongate filaments have a first end and a second end, wherein the first and second ends of each of the plurality of elongate filaments are gathered at the proximal end of the permeable shell in a proximal band, and wherein the transitional section of the permeable shell comprises a first end portion connected to a distal end of the first layer and a second end portion connected to a distal end of the second layer, wherein the second end portion is inverted, and wherein the transitional portion of the plurality of filaments is not surrounded by a band.

In some embodiments, the proximal band is the only band in the device.

In some embodiments, the second layer is an inner layer of the device and the first layer is an outer layer of the device.

In some embodiments, the transitional section comprises a height that is less that about ¼ of a total height of the permeable shell.

In some embodiments, in an unconstrained configuration, an expanded shape of the first layer is different than an expanded shape of the second layer.

In some embodiments, in an unconstrained configuration, an outer surface of a distal region of an expanded shape of the first layer has a substantially frustoconical shape.

In some embodiments, in an unconstrained configuration, the second layer comprises an expanded shape comprising a top surface that defines a plane that is substantially perpendicular to a vertical axis of the permeable shell.

In some embodiments, in an unconstrained configuration, an inner surface of a distal portion of the expanded shape of the first layer does not contact at least about 50% of a first area defined by the plane of the top surface.

In some embodiments, in an unconstrained configuration, the expanded shape of the second layer is substantially barrel shape.

In some embodiments, in an unconstrained configuration, the second layer comprises an expanded shape comprising a proximal portion having a substantially frustoconical shape, wherein the proximal portion of having the substantially frustoconical shape defines a second area. In some embodiments, an inner surface of a proximal portion of the expanded shape of the first layer does not contact at least about 50% of the second area.

In some embodiments, the mesh is a tubular mesh having an inner surface and an outer surface, wherein an outer surface of the first layer is the inner surface of the tubular mesh.

In some embodiments, in an unconstrained configuration, the permeable mesh further comprises a distal void space in a distal region of the permeable shell and a proximal void space in the proximal region of the permeable shell. In some embodiments, the distal void space is larger than the proximal void space. In some embodiments, the distal void space is defined by a top surface of the expanded state of the second layer, an outer surface of the elongate section, and an inner surface of a distal region of the first layer. In some embodiments, the proximal void space is defined by a bottom surface of an expanded state of the second layer and an inner surface of a proximal region of the first layer.

In some embodiments, in an unconstrained configuration, the first layer does not have a corrugated or undulating portion.

In some embodiments, in an unconstrained configuration, the second layer does not have a corrugated or undulating portion.

In many embodiments, a method for treating a cerebral aneurysm having an interior cavity and a neck includes the steps of: advancing an implant in a microcatheter to a region of interest in a cerebral artery, wherein the implant comprises: a permeable shell comprising a first layer, a second layer, a proximal end, a distal end, and a transitional section connecting the first and second layer, wherein the permeable shell has a radially constrained elongated state configured for delivery within a lumen of the microcatheter and an expanded state with a longitudinally shortened configuration relative to the radially constrained state, wherein the first and second layers are formed from a plurality of elongate filaments that are woven together to form a mesh, wherein each of the plurality of elongate filaments have a first end and a second end, wherein the first and second ends of each of the plurality of elongate filaments are gathered at the proximal end of the permeable shell in a proximal band, and wherein the transitional section of the permeable shell comprises a first end portion connected to a distal end of the first layer and a second end portion connected to a distal end of the second layer, wherein the second end portion is inverted, and wherein the intermediate portion of the plurality of filaments is not surrounded by a band; deploying the implant within the cerebral aneurysm, wherein the permeable shell expands to the expanded state in the interior cavity of the aneurysm; and withdrawing the microcatheter from the region of interest after deploying the implant.

In some embodiments, the proximal band is the only band in the device.

In some embodiments, the second layer is an inner layer of the device, and the first layer is an outer layer of the device. In some embodiments, the mesh is inverted at the distal end of the permeable shell to form the first and second layers of the permeable shell. In some embodiments, the mesh is a tubular mesh having an inner surface and an outer surface, wherein an outer surface of the first layer is the inner surface of the tubular mesh.

### BRIEF DESCRIPTION OF THE DRAWINGS

These and other aspects, features, and advantages of which embodiments of the invention are capable of will be apparent and elucidated from the following description of embodiments of the present invention, reference being made to the accompanying drawings, in which:

The various figures included show the occlusive device according to one or more embodiments.

FIGS. 1A-1B depict exemplary multi-layer vascular implants.

FIG. 2 depicts an exemplary multi-layer vascular implant deployed in an aneurysm.

### DESCRIPTION OF EMBODIMENTS

The presented embodiments shall generally relate to occlusive devices that include multiple layers and achieve the desired characteristics of being deliverable with low force through small microcatheters, having a high enough radial force to fully deploy and remain positioned within the aneurysm after detachment, and having a high metal surface area for flow stasis.

Discussed herein are devices and methods for the treatment of vascular defects that are suitable for minimally invasive deployment within a patient's vasculature, and particularly, within the cerebral vasculature of a patient. For such embodiments to be safely and effectively delivered to a desired treatment site and effectively deployed, some device embodiments may be configured for collapse to a low-profile constrained state with a transverse dimension suitable for delivery through an inner lumen of a microcatheter and deployment from a distal end thereof.

Embodiments of these devices may also maintain a clinically effective configuration with sufficient mechanical integrity once deployed so as to withstand dynamic forces within a patient's vasculature over time that may otherwise result in compaction of a deployed device. It may also be desirable for some device embodiments to occlude a vascular defect of a patient acutely during the course of a procedure in order to provide more immediate feedback regarding success of the treatment to a treating physician.

Intrasaccular occlusive devices that include a permeable shell formed from a woven or braided mesh have been described in U.S. 2017/0095254, US 2016/0249934, US 2016/0367260, U.S. 2016/0249937, and U.S. 2018/0000489, all of which are hereby expressly incorporated by reference in their entireties for all purposes.

Some embodiments are particularly useful for the treatment of cerebral aneurysms by reconstructing a vascular wall so as to wholly or partially isolate a vascular defect from a patient's blood flow. Some embodiments may be configured to be deployed within a vascular defect to facilitate reconstruction, bridging of a vessel wall or both in order to treat the vascular defect. For some of these embodiments, the permeable shell of the device may be configured to anchor or fix the permeable shell in a clinically beneficial position. For some embodiments, the device may be disposed in whole or in part within the vascular defect in order to anchor or fix the device with respect to the vascular structure or defect. The permeable shell may be configured to span an opening, neck or other portion of a vascular defect in order to isolate the vascular defect, or a portion thereof, from the patient's nominal vascular system in order allow the defect to heal or to otherwise minimize the risk of the defect to the patient's health.

Occlusion implants made from single layer braids that incorporate a mixture of filament sizes. Occlusion implants may also be made from single layer braids that only incorporate a single filament size. The wires or filaments used to make the mesh braids may be made of super-elastic (e.g., nitinol) and/or composite super-elastic/radiopaque (nitinol-platinum “DFT”) material.

In some embodiments, the single layer braids may be folded over and inverted to create a double layer or multiple layer implant. The additional layer of the implant may provide increased structural support. The additional layer may also increase the metal surface area, thereby improving flow stasis in the cavity of the aneurysm.

The plurality of filaments that make up the mesh may be made from nitinol, stainless steel, drawn filled tubing (e.g., platinum or tantalum core with a nitinol jacket), platinum, platinum alloys such as platinum/tungsten, or a mixture thereof. The number of filaments may be between 4 to about 216 wires, alternatively between about 32 to about 216 wires. Suitable materials and numbers of filaments for constructing mesh implants are described in U.S. 2017/0095254, U.S. 2016/0249934, U.S. 2016/0367260, U.S. 2016/0249937, and U.S. 2018/0000489, all of which are hereby expressly incorporated by reference in their entireties for all purposes.

The use of multiple marker bands in occlusion implants (e.g., one proximal marker band and one or more distal marker bands (e.g., one for each layer)) can be problematic because the middle marker band may not be perfectly aligned with the distal marker band, resulting in asymmetric collapse (“cobra-heading”) and/or protrusion of the middle marker band through the outer layer. Incorporating three marker bands is also more difficult during manufacturing because it requires more assembly steps.

Note though the term “marker bands” are used herein to discuss various embodiments, these elements are configured as hubs which are generally a junction where the wires forming a device, device section, or device layer are attached together along the hub (e.g., cylindrical structure). Often it is beneficial that this hub is radiopaque to aid in visualization, and as such the hub often takes on the form of a marker band. In this way, the marker band is inclusive of a hub concept.

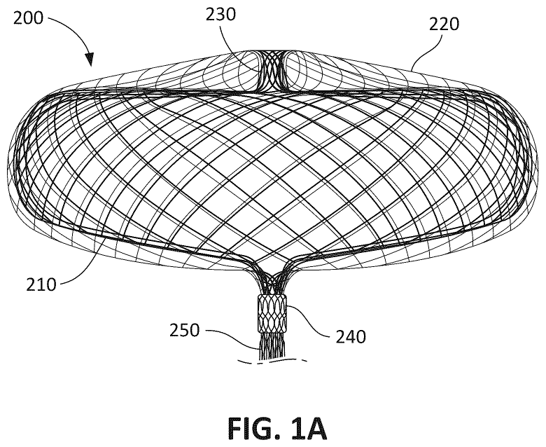

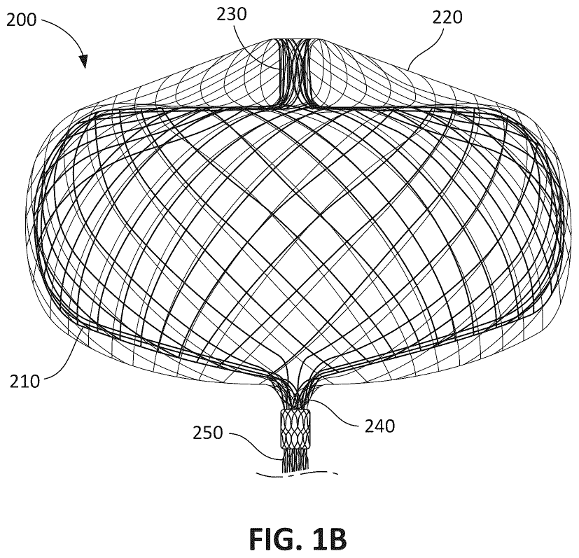

In some embodiments, as seen in FIGS. 1A-1B, the dual layer implant 200 includes an inner layer and an outer layer. The implant may be made from a single tubular mesh of braided filaments that is inverted in a middle or transitional portion, where the inverted part of the tubular mesh becomes the distal region or distal end of the implant. Each of the plurality of filaments making up the tubular mesh may have a first end and a second end. Both of the first and second ends of each of the plurality of filaments may be gathered in a single hub or marker band 240 at the proximal end of the implant 200. The single hub or marker band may be detachably connected to a pusher 250. In some embodiments, the implant 200 does not include a distal marker band.

The mesh may be heat-set such that in the expanded state, the implant 200 includes a first inner mesh layer 210 and a second outer mesh layer 220 that are connected by an elongate transition region 230. The elongate transition region 230 may not include a band or marker around the filaments. A height of the elongate transition region 230 may be less than about ¼, alternatively less than about ⅓, alternatively less than about ⅕, alternatively less than about ⅙ of the total height of the permeable shell. The elongate transition region 230 may have a smaller diameter than an unconstrained, expanded diameter of the first and or second mesh layers 210, 220. The diameter of the elongate transition region may be substantially similar to a diameter of the first and second ends of the plurality of filaments gathered in the hub 240.

Either layer of the occlusion implants of the embodiments described in this application may have expanded states in unconstrained configurations that can have a substantially globular, spherical, or cylindrical shape. Alternatively, the expanded state can have a shape similar to a pumpkin. The proximal and/or distal ends may be recessed or may be substantially flat. The proximal and/or distal ends may have a frustoconical or substantially frustoconical shape. The distal and proximal ends of the occlusion implants (e.g., where the wires bend (e.g., between about 90°-about 120°) and transition from the substantially flat distal and proximal ends to the middle region) contribute nearly all of the radial stiffness to the implant, wherein the middle region (forming the substantially vertical or partially curved walls of the expanded state) are almost immeasurably soft.

In some embodiments, in an unconstrained configuration, the inner layer may have an expanded state that is substantially barrel-shaped or disc shaped, where the disc has a height that is between approximately 50-90%, alternatively between approximately 50-80%, of the height of the expanded state of the outer layer.

In some embodiments, in an unconstrained configuration, the inner layer may have an expanded state that has a top surface that defines an area. In some embodiments, the top surface may define a plane that is substantially perpendicular to a vertical axis of the permeable shell. In some embodiments, an inner surface of a distal portion of the expanded shape of the outer layer does not contact at least about 50% of the area defined by the plane of the top surface if the expanded state of the inner layer.

In some embodiments, in an unconstrained configuration, the inner layer may have an expanded state that has a bottom surface that defines an area. In some embodiments, the bottom surface has a substantially frustoconical shape defining an area. In some embodiments, an inner surface of a proximal portion of the expanded shape of the outer layer does not contact at least about 50% of the area defined by the frustoconical shape of the bottom surface.

In some embodiments, in an unconstrained configuration, the inner layer may have a smooth curved surface. The expanded shape of the inner layer may not have a corrugated or undulating portion.

In some embodiments, in an unconstrained configuration, the outer layer may have a smooth curved surface. The expanded shape of the outer layer may not have a corrugated or undulating portion.

In some embodiments, the implants in their expanded or heat set state before being deployed in an aneurysm (i.e., unconstrained configuration) include a void space in the distal region and a void space in the proximal region. The void space in the distal region of the permeable shell may be defined by the top surface of the expanded state of the inner layer, the outer surface of the transitional region, and the inner surface of the distal region of the outer layer. The void space in the proximal region of the permeable shell may be defined by the bottom surface of the expanded state of the inner layer and the inner surface of the proximal region of the outer layer. The void space in the distal region may be larger than the void space in the proximal region. A large distal void space may allow for the implant to conform more to the dome of the aneurysm without damaging the dome.

In some embodiments, the outer layer may have an expanded shape having a proximal region and a distal region. The distal region of the expanded state of the outer layer may have a frustoconical shape or a roughly or substantially frustoconical shape.

Generally speaking, in order to prepare a multi-layer implant 200, the layers 210, 220 of the permeable shell must first be formed. In order to shape these layers, various molds (e.g., two molds) each having a unique shape corresponding to each separate layer are used. In order to create this bi-layer design, one or more tubular bands may be used to create a constriction between the two layers, thereby creating the transitional section. Generally, a tubular mesh is used as a basic implant layer, where the mesh and band as described are then placed over the various mold shapes to create different shaped sections. Where the tubular bands as described are used to create transition sections, the sections can then be enveloped backwards to create a bi-layer layer implant. The tubular bands may then be removed such that no band surrounds the transitional section in the final implant.

### DELIVERY OF OCCLUSIVE IMPLANTS TO ANEURYSMS

All of the embodiments of permeable shells described herein have a radially constrained elongated state configured for delivery within a microcatheter, and an expanded relaxed state with a globular or barrel-like, longitudinally shortened configuration relative to the radially constrained state. In the expanded state, the permeable shell can have a maximum diameter of about 4 mm, alternatively about 5 mm, alternatively about 6 mm, alternatively about 7 mm, alternatively about 8 mm, alternatively about 9 mm, alternatively about 10 mm, alternatively about 11 mm. The expanded state of the permeable shell can have a height or length of about 2.6 mm, about 3 mm, about 3.6 mm, about 4 mm, about 4.6 mm, about 5 mm, about 5.6 mm, about 6 mm, about 6.6 mm, about 7 mm, about 7.6 mm, about 8 mm, about 8.6 mm, about 9 mm, about 9.6 mm, or about 10 mm. The woven structure of the filaments forming the mesh portions includes a plurality of openings in the permeable shell formed between the woven filaments. In some embodiments, the occlusive device can be configured as an intrasaccular occlusive device which generally conforms to the shape of the treatment site. The permeable shells may have an expanded shape that is substantially globular, spherical, cylindrical, or shaped similar to a pumpkin.

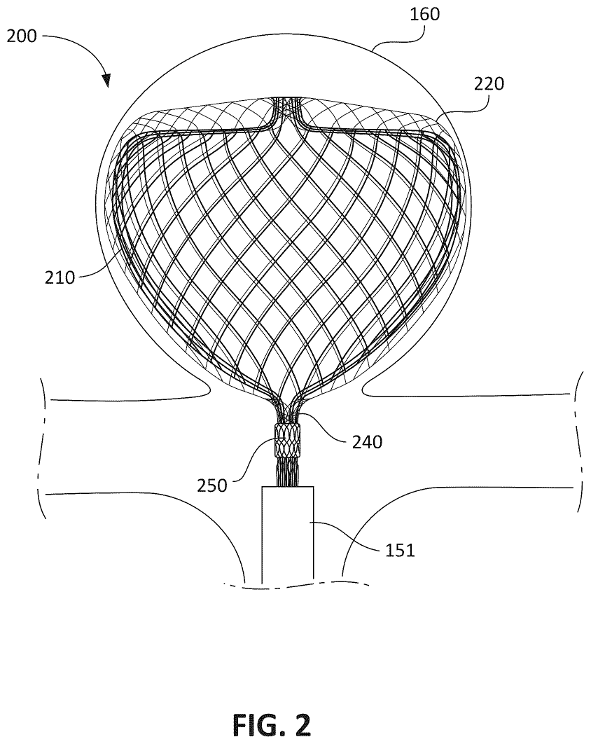

As seen in FIG. 2, the permeable shell can be delivered by advancing a pusher detachably coupled to a proximal end of the permeable shell through a lumen of the microcatheter 151. Once it has been deployed in the region of interest (inner cavity of the aneurysm 160), the permeable shell 200 can be detached from the pusher 151. The implant 200 may conform to the shape of the inner cavity of the aneurysm 160. The implant 200 may be detachably coupled to the distal end of the pusher through a thermal, mechanical, or electrolytic mechanism.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that the feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

### Clauses

Exemplary embodiments are set out in the following numbered clauses.

Clause 1. A device for treatment of a patient's cerebral aneurysm, comprising:
a permeable shell comprising a first layer, a second layer, a proximal end, a distal end, and a transitional section connecting the first and second layer, wherein the permeable shell has a radially constrained elongated state configured for delivery within a catheter lumen and an expanded state with a longitudinally shortened configuration relative to the radially constrained state,
wherein the first and second layers are formed from a plurality of elongate filaments that are woven together to form a mesh, wherein each of the plurality of elongate filaments have a first end and a second end, wherein the first and second ends of each of the plurality of elongate filaments are gathered at the proximal end of the permeable shell in a proximal band, and
wherein the transitional section of the permeable shell comprises a first end portion connected to a distal end of the first layer and a second end portion connected to a distal end of the second layer, wherein the second end portion is inverted, and wherein the transitional portion of the plurality of filaments is not surrounded by a band.

Clause 2. The device of clause 1, wherein the proximal band is the only band in the device.

Clause 3. The device of clause 1, wherein the second layer is an inner layer of the device and the first layer is an outer layer of the device.

Clause 4. The device of clause 1, wherein the transitional section comprises a height that is less that about ¼ of a total height of the permeable shell.

Clause 5. The device of clause 1, wherein in an unconstrained configuration, an expanded shape of the first layer is different than an expanded shape of the second layer.

Clause 6. The device of clause 1, wherein in an unconstrained configuration, an outer surface of a distal region of an expanded shape of the first layer has a substantially frustoconical shape.

Clause 7. The device of clause 1, wherein in an unconstrained configuration, the second layer comprises an expanded shape comprising a top surface that defines a plane that is substantially perpendicular to a vertical axis of the permeable shell.

Clause 8. The device of clause 7, wherein in an unconstrained configuration, an inner surface of a distal portion of the expanded shape of the first layer does not contact at least about 50% of a first area defined by the plane of the top surface.

Clause 9. The device of clause 7, wherein in an unconstrained configuration, the expanded shape of the second layer is substantially barrel shape.

Clause 10. The device of clause 1, wherein in an unconstrained configuration, the second layer comprises an expanded shape comprising a proximal portion having a substantially frustoconical shape, wherein the proximal portion of having the substantially frustoconical shape defines a second area.

Clause 11. The device of clause 10, wherein an inner surface of a proximal portion of the expanded shape of the first layer does not contact at least about 50% of the second area.

Clause 12. The device of clause 1, wherein the mesh is a tubular mesh having an inner surface and an outer surface, wherein an outer surface of the first layer is the inner surface of the tubular mesh.

Clause 13. The device of clause 1, wherein in an unconstrained configuration, the permeable mesh further comprises a distal void space in a distal region of the permeable shell and a proximal void space in the proximal region of the permeable shell.

Clause 14. The device of clause 13, wherein the distal void space is larger than the proximal void space.

Clause 15. The device of clause 13, wherein the distal void space is defined by a top surface of the expanded state of the second layer, an outer surface of the elongate section, and an inner surface of a distal region of the first layer.

Clause 16. The device of clause 13, wherein the proximal void space is defined by a bottom surface of an expanded state of the second layer and an inner surface of a proximal region of the first layer.

Clause 17. The device of clause 1, wherein in an unconstrained configuration, the first layer does not have a corrugated or undulating portion.

Clause 18. The device of clause 1, wherein in an unconstrained configuration, the second layer does not have a corrugated or undulating portion.

Clause 19. A method for treating a cerebral aneurysm having an interior cavity and a neck, comprising the steps of:
advancing an implant in a microcatheter to a region of interest in a cerebral artery, wherein the implant comprises:
a permeable shell comprising a first layer, a second layer, a proximal end, a distal end, and a transitional section connecting the first and second layer, wherein the permeable shell has a radially constrained elongated state configured for delivery within a lumen of the microcatheter and an expanded state with a longitudinally shortened configuration relative to the radially constrained state,
   wherein the first and second layers are formed from a plurality of elongate filaments that are woven together to form a mesh, wherein each of the plurality of elongate filaments have a first end and a second end, wherein the first and second ends of each of the plurality of elongate filaments are gathered at the proximal end of the permeable shell in a proximal band, and
   wherein the transitional section of the permeable shell comprises a first end portion connected to a distal end of the first layer and a second end portion connected to a distal end of the second layer, wherein the second end portion is inverted, and wherein the intermediate portion of the plurality of filaments is not surrounded by a band;
deploying the implant within the cerebral aneurysm, wherein the permeable shell expands to the expanded state in the interior cavity of the aneurysm; and
   withdrawing the microcatheter from the region of interest after deploying the implant.

Clause 20. The method of clause 19, wherein the proximal band is the only band in the device.

Clause 21. The method of clause 19, wherein the second layer is an inner layer of the device and the first layer is an outer layer of the device.

Clause 22. The method of clause 19, wherein the mesh is inverted at the distal end of the permeable shell to form the first and second layers of the permeable shell.

Clause 23. The method of clause 19, wherein the mesh is a tubular mesh having an inner surface and an outer surface, wherein an outer surface of the first layer is the inner surface of the tubular mesh.

1. A device for treatment of a patient's cerebral aneurysm, comprising:
a permeable shell comprising a first layer, a second layer, a proximal end, a distal end, and a transitional section connecting the first and second layer, wherein the permeable shell has a radially constrained elongated state configured for delivery within a catheter lumen and an expanded state with a longitudinally shortened configuration relative to the radially constrained state,
wherein the first and second layers are formed from a plurality of elongate filaments that are woven together to form a mesh, wherein each of the plurality of elongate filaments have a first end and a second end, wherein the first and second ends of each of the plurality of elongate filaments are gathered at the proximal end of the permeable shell in a proximal band,
wherein the transitional section of the permeable shell comprises a first end portion connected to a distal end of the first layer and a second end portion connected to a distal end of the second layer, wherein the second end portion is inverted, and wherein the transitional portion of the plurality of elongate filaments is not surrounded by a band,
wherein in an unconstrained configuration, the permeable shell further comprises a distal void space in a distal region of the permeable shell and a proximal void space in the proximal region of the permeable shell, and wherein the proximal void space is defined by a bottom surface of an expanded state of the second layer and an inner surface of a proximal region of the first layer.

2. The device of claim 1, wherein the proximal band is the only band in the device.

3. The device of claim 1, wherein the second layer is an inner layer of the device and the first layer is an outer layer of the device.

4. The device of claim 1, wherein the transitional section comprises a height that is less than about ¼ of a total height of the permeable shell.

5. The device of claim 1, wherein in the unconstrained configuration, an expanded shape of the first layer is different than an expanded shape of the second layer.

6. The device of claim 1, wherein in the unconstrained configuration, an outer surface of a distal region of an expanded shape of the first layer has a substantially frustoconical shape.

7. The device of claim 1, wherein in the unconstrained configuration, the second layer comprises an expanded shape comprising a top surface that defines a plane that is substantially perpendicular to a vertical axis of the permeable shell.

8. The device of claim 7, wherein in the unconstrained configuration, an inner surface of a distal portion of the expanded shape of the first layer does not contact at least about 50% of a first area defined by the plane of the top surface.

9. The device of claim 7, wherein in the unconstrained configuration, the expanded shape of the second layer is substantially a barrel shape.

10. The device of claim 1, wherein in the unconstrained configuration, the second layer comprises an expanded shape comprising a proximal portion having a substantially frustoconical shape, wherein the proximal portion having the substantially frustoconical shape defines a second area.

11. The device of claim 10, wherein an inner surface of a proximal portion of the expanded shape of the first layer does not contact at least about 50% of the second area.

12. The device of claim 1, wherein the mesh is a tubular mesh having an inner surface and an outer surface, wherein an outer surface of the first layer is the inner surface of the tubular mesh.

13. (canceled)

14. The device of claim 1, wherein the distal void space is larger than the proximal void space.

15. The device of claim 1, wherein the distal void space is defined by a top surface of the expanded state of the second layer, an outer surface of the elongate section, and an inner surface of a distal region of the first layer.

16. (canceled)

17. The device of claim 1, wherein in the unconstrained configuration, the first layer does not have a corrugated or undulating portion.

18. The device of claim 1, wherein in the unconstrained configuration, the second layer does not have a corrugated or undulating portion.

19. A method for treating a cerebral aneurysm having an interior cavity and a neck, comprising the steps of:
advancing an implant in a microcatheter to a region of interest in a cerebral artery, wherein the implant comprises:
a permeable shell comprising a first layer, a second layer, a proximal end, a distal end, and a transitional section connecting the first and second layer, wherein the permeable shell has a radially constrained elongated state configured for delivery within a lumen of the microcatheter and an expanded state with a longitudinally shortened configuration relative to the radially constrained state,
wherein the first and second layers are formed from a plurality of elongate filaments that are woven together to form a mesh, wherein each of the plurality of elongate filaments have a first end and a second end, wherein the first and second ends of each of the plurality of elongate filaments are gathered at the proximal end of the permeable shell in a proximal band, and
wherein the transitional section of the permeable shell comprises a first end portion connected to a distal end of the first layer and a second end portion connected to a distal end of the second layer, wherein the second end portion is inverted, and wherein the intermediate portion of the plurality of elongate filaments is not surrounded by a band, wherein in an unconstrained configuration, the permeable shell further comprises a distal void space in a distal region of the permeable shell and a proximal void space in the proximal region of the permeable shell, and wherein the proximal void space is defined by a bottom surface of an expanded state of the second layer and an inner surface of a proximal region of the first layer;
deploying the implant within the cerebral aneurysm, wherein the permeable shell expands to the expanded state in the interior cavity of the aneurysm; and
withdrawing the microcatheter from the region of interest after deploying the implant.

20. The method of claim 19, wherein the proximal band is the only band in the implant.

21-23. (canceled)

24. The device of claim 1, wherein the distal and proximal ends are connected to a middle region of the permeable shell by portions of the plurality of elongate filaments bent at angles between about 90° and about 120°.

25. The device of claim 24, wherein the distal and proximal ends have greater radial stiffness than the middle region of the permeable shell.
